(19) United States
(12) Patent Application Publication
(10) Pub. No.: US 2019/0107641 A1
(43) Pub. Date: Apr. 11, 2019

(54) SENSING SYSTEM FOR AN OCCUPANT SUPPORT

(71) Applicant: Faurecia Automotive Seating, LLC, Auburn Hills, MI (US)

(72) Inventor: John M. PERRAUT, Rochester Hill, MI (US)

(21) Appl. No.: 16/149,716

(22) Filed: Oct. 2, 2018

Related U.S. Application Data
(60) Provisional application No. 62/568,347, filed on Oct. 5, 2017.

(51) Int. Cl. G01V 3/38; B60N 2/00; G01V 3/08; A61B 5/11; A61B 5/00; A61B 5/024; A61B 5/08

(52) U.S. Cl. CPC G01V 3/38; B60N 2/002; G01V 3/08; A61B 5/1113; A61B 5/0816; A61B 5/1116; A61B 5/1102; A61B 5/6893; A61B 5/024; A61B 5/1126

(57) ABSTRACT

An occupant support includes a vehicle seat and a sensor system coupled to the vehicle seat. The sensor system is configured to measure capacitance data of an occupant of the vehicle seat.

### PRIORITY CLAIM

This application claims priority under 35 U.S.C. § 119(e) to U.S. Provisional Application Ser. No. 62/568,347, filed Oct. 5, 2017, which is expressly incorporated by reference herein.

### BACKGROUND

The present disclosure relates to sensor systems for use with occupant supports. More particularly, the present disclosure relates to capacitive sensor systems.

### SUMMARY

According to the present disclosure, an occupant support includes a vehicle seat and a sensor system. The vehicle seat includes a plurality of capacitive sensors, wherein the capacitive sensors includes a first transmitter and a first receiver.

In illustrative embodiments, the sensor system includes a controller and an analog interface circuit coupled to the plurality of capacitive sensors. The sensor system is configured to transmit an excitation signal to the first transmitter, wherein the first transmitter and the first receiver establish a fringe field in response to transmission of the excitation signal, receive a capacitance signal from the first receiver in response to transmission of the excitation signal, and determine whether an occupant is present in the vehicle seat as a function of the capacitance signal based on capacitive coupling between the occupant and the fringe field in response to receipt of the capacitance signal. In illustrative embodiments, the analog interface circuit includes an analog to digital converter or a capacitance to digital converter.

In illustrative embodiments, the vehicle seat includes a trim layer and a carrier layer, wherein the trim layer covers the carrier layer, and wherein the carrier layer includes the plurality of capacitive sensors. In illustrative embodiments, the carrier layer includes a heat mat that generates heat in response to application of a power signal, wherein the power signal is combined with the excitation signal. In illustrative embodiments, each of the plurality of capacitive sensors includes a piezoelectric film.

In illustrative embodiments, to determine whether the occupant is present further includes to determine a position of the occupant relative to the vehicle seat. In illustrative embodiments, to determine the position of the occupant includes to determine a three-dimensional position of the occupant.

In illustrative embodiments, the sensor system is further configured to determine biometric data associated with the occupant as a function of the capacitance signal. In illustrative embodiments, the biometric data includes a body morphological measurement of the occupant. In illustrative embodiments, the biometric data comprises ballistocardiograph data. In illustrative embodiments, to determine whether the occupant is present in the vehicle seat as a function of the capacitance signal includes to determine whether the occupant is present based on coupling of the fringe field to a body of the occupant in a shunt mode or in a transmit mode.

According to another aspect of the present disclosure, an occupant support includes a vehicle seat and a sensor system. The vehicle seat includes a first transmitter and a first receiver. The first transmitter and the first receiver are positioned close together on the vehicle seat.

In illustrative embodiments, the sensor system includes a controller and an analog interface circuit coupled to the first transmitter and the first receiver. The sensor system is configured to transmit an excitation signal to the first transmitter, wherein the first transmitter and the first receiver establish a near field in response to transmission of the excitation signal, receive a capacitance signal from the first receiver in response to transmission of the excitation signal, wherein the capacitance signal fluctuates based on mechanical distortion of the vehicle seat caused by an occupant of the vehicle seat, and determine ballistocardiograph data as a function of the capacitance signal in response to receipt of the capacitance signal. In illustrative embodiments, the ballistocardiograph data may be indicative of heart rate of the occupant or breathing rate of the occupant.

Additional features of the present disclosure will become apparent to those skilled in the art upon consideration of illustrative embodiments exemplifying the best mode of carrying out the disclosure as presently perceived.

### BRIEF DESCRIPTIONS OF THE DRAWINGS

The detailed description particularly refers to the accompanying figures in which:

FIG. 1 is a perspective and diagrammatic view of a sensor system in accordance with the present disclosure coupled to an occupant support suggesting that the sensor system includes a plurality of sensors configured to measure biometric data of an occupant positioned on the occupant support and a control system coupled to the sensors;

FIG. 2 is a rear perspective view of the sensor system of FIG. 1 coupled to the occupant support showing the control system housed in a back of the occupant support;

FIG. 3 is a diagrammatic side view of the sensor system of FIGS. 1 and 2;

FIG. 4 is a diagrammatic side view of the sensor system of FIGS. 1 and 2 operating in a shunt mode;

FIG. 5 is a diagrammatic side view of the sensor system of FIGS. 1 and 2 operating in a transmit mode;

FIG. 6 is a simplified block diagram of the sensor system of FIGS. 1-5;

FIG. 7 is a plot of an excitation signal that may be generated by the sensor system of FIGS. 1-6; and

FIG. 8 is a simplified block diagram illustrating an environment that may be established by a controller of the sensor system of FIGS. 1-7.

### DETAILED DESCRIPTION

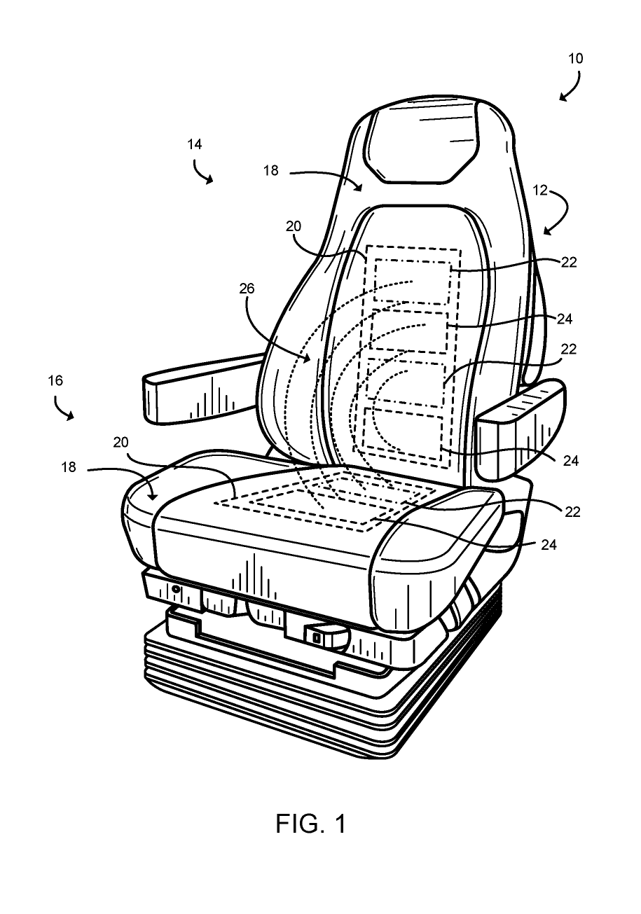

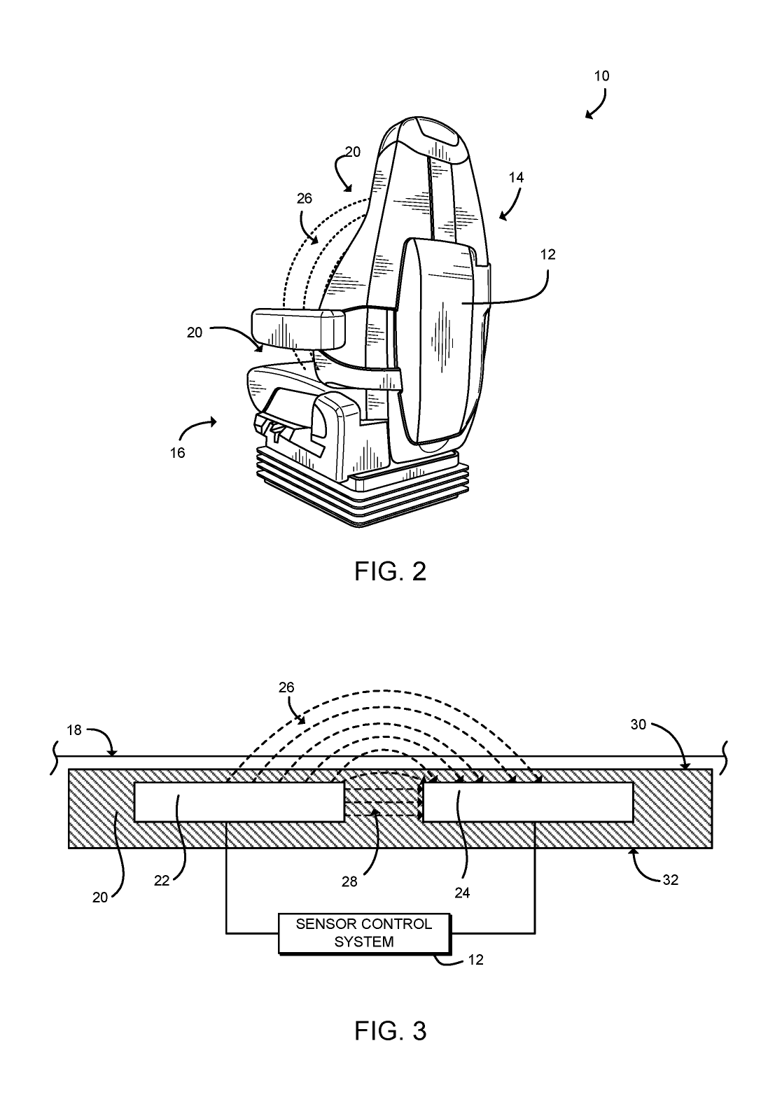

A sensor control system 12 in accordance with the present disclosure is adapted for use with an occupant support 10 such as, for example, a seat as shown in FIGS. 1 and 2. Occupant support 10 may be included in a vehicle or occupant support 10 may be any occupant support 10 configured to support an occupant.

As shown in FIGS. 1 and 2, the occupant support 10 includes a seat back 14 and a seat bottom 16. Both the seat back 14 and the seat bottom 16 are covered with a trim cover 18. The trim cover 18 may be embodied as a fabric, mesh, or any other suitable covering of the occupant support 10. Each of the seat back 14 and the seat bottom 16 includes, for example, a heating mat 20 underneath the trim cover 18. The heating mat 20 generates heat or a radiated electromagnetically compatible signal in response to application of a power signal. The power signal may be embodied as DC current, AC current, or a combination of DC and AC signals. The heating mat 20 may be positioned on top of, within, or below one or more other layers of the occupant support 10, such as one or more comfort layers (e.g., foam layers).

As shown, each heating mat 20 includes multiple capacitive sensors 22, 24. As described further below, illustratively the sensors 22 are transmitters and the sensors 24 are receivers. Each sensor 22, 24 may be embodied as a conductive plate, film, wire, or other electrode capable of transmitting and/or receiving an electric field. Each sensor 22, 24 is illustratively embedded in a heating mat 20. In other embodiments the sensors 22, 24 may be included in the trim cover 18, a comfort layer, or other carrier layer of the occupant support 10.

The sensors 22, 24 may be arranged on the occupant support 10 to facilitate detecting the location of the occupant. In some examples, the sensors 22, 24 may be arranged in a grid over occupant support, for example a two-dimensional grid of wires, yarns, or other electrodes near the trim cover 18 surface. In some other examples, the types of heating mats 20 and/or sensors 22, 24 may vary, including the orientation, configuration, number, shape, and/or application in a vehicle seat. Additionally, although illustrated as including multiple transmitters 22 and receivers 24, it should be understood that in some embodiments the occupant support 10 may include multiple transmitters 22 and a single receiver 24 or another arrangement of sensors 22, 24.

In some examples, each of the sensors 22, 24 may be embodied as a piezoelectric film transducer. The piezoelectric film transducer material may be a polymer material such as polyvinylidene fluoride (PVDF), with softness that does not compromise occupant comfort, and may be highly reliable and thereby suitable for repeated use. The piezoelectric film transducer material may be coupled to the occupant seat 10, for example by being clamped on either end of a top surface of an internal comfort layer of the occupant support 10 (e.g., a surface of a seat cushion closest to the trim cover 18). In addition to occupant detection, the piezoelectric film transducer may detect fluctuations in capacitive signal due to rhythmic heart rate, breathing, and/or movement of the occupant. A low-frequency band pass signal may be used to detect fluctuation associated with biometric data (e.g, ballistocardiograph data).

As described further below, in operation, the sensors 22, 24 generate a fringe field 26 extending above the trim cover 18. When an occupant's body is positioned on the occupant support 10, the occupant's body may interact with the fringe field 26 to shield, block, transmit, fluctuate, or otherwise modify the fringe field 26. As described further below, the sensor control system 12 is configured to detect the presence of the occupant, the position of the occupant, and/or biometric data of the occupant based on those interactions with the fringe field 26.

Referring now to FIGS. 3-5, generation of the fringe field 26 and interaction with the occupant of the occupant support 10 are illustrated. As shown, the sensor control system 12 is coupled to the sensors 22, 24. The sensors 22, 24 are embedded within the heat mat 20. As shown, the heat mat 20 has a top surface 30 close to the trim cover 18 and a bottom surface 32. The sensors 22, 24 may be embedded in the heat map 20 on of or otherwise in proximity to the top surface 30. The sensor control system 12 applies an excitation signal to the transmitter 22. The excitation signal is illustratively an AC signal combined with a DC offset. In other embodiments, the excitation signal may be embodied as a DC signal, an AC signal, or other signal. The AC component of the excitation signal, for example, may be frequency modulated or amplitude modulated. One potential example of the excitation signal is illustrated in FIG. 7 and described further below.

Applying the excitation signal causes a confined field 28 between the transmitter 22 and the receiver 24. The confined field 28 may be embodied as an electrical field, including a constant electrical field, a varying electric field, or other field. Applying the excitation signal also causes the fringe field 26 to extend from the transmitter 22 to the receiver 24, beyond the trim cover 18.

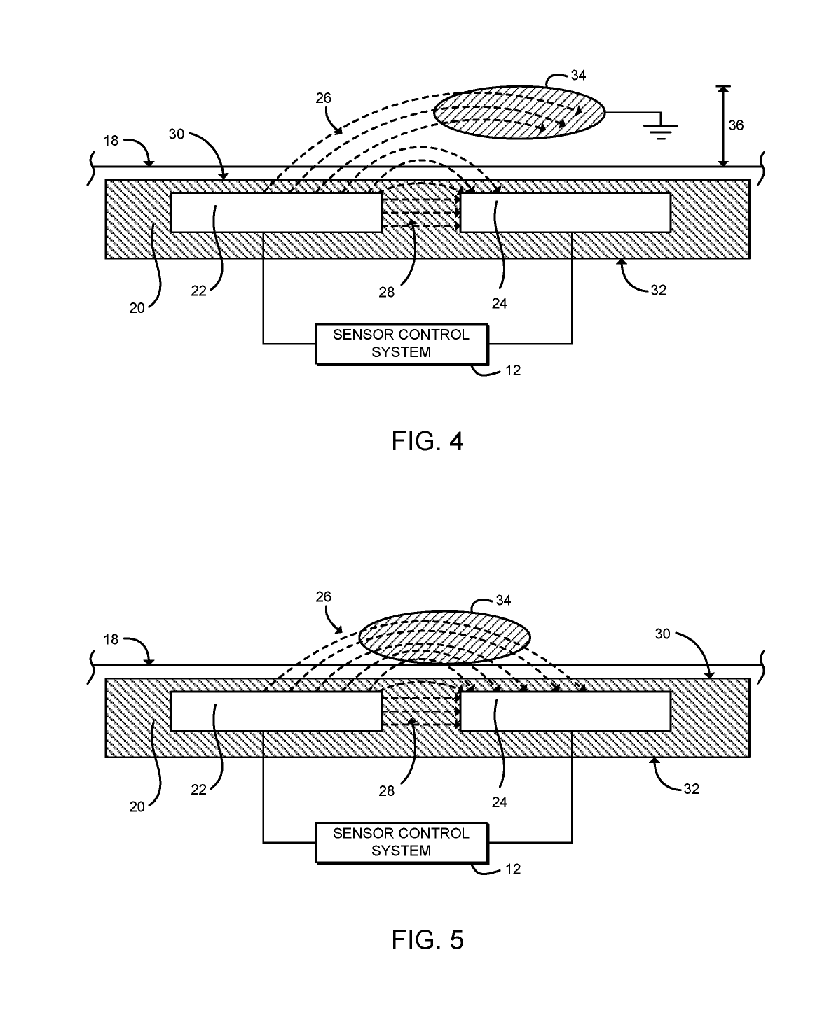

A body 34 of the occupant of the occupant support 10 may interact with the fringe field 26 in a shunt mode as shown in FIG. 4. In shunt mode, the body 34 of the occupant (or a part of the body 34) absorbs, blocks, and/or shields part of the fringe field 26, for example by shunting part of the fringe field 26 to ground. As shown, in shunt mode the body 34 may be positioned at a range 36 from the trim cover 18 and/or the sensors 22, 24. The range 36 may be between zero and about four inches (about ten centimeters). Thus, in the shunt mode, the presence of the body 34 at the range 36 may change capacitance between the transmitter 22 and the receiver 24 and thus change the signal received by the receiver 24. For example, the capacitance of the occupant may be sensed as a decrease in coupling between local electrodes (e.g., the transmitter 22 and receiver 24) thereby causing a shielding effect of relatively high capacitance of the occupant's body 34. Because the distance between the sensors 22, 24 is known, morphological or other biometric measurements of the occupant may be derived from the received signal.

The body 34 of the occupant of the occupant support 10 may interact with the fringe field 26 in a transmit mode as shown in FIG. 5. In transmit mode, the body 34 of the occupant (or a part of the body 34) transmits part of the fringe field 26 to the receiver 24, without blocking or shielding the fringe field 26. Thus, in the transmit mode the body 34 of the user effectively becomes an extension of the transmitter 22. In the transmit mode, the fringe field 26 is transmitted from the transmitter 22 to the receiver 24 without a connection to ground. In the transmit mode, the body 34 may be very close to the sensors 22, 24, for example in contact with the trim cover 18 as shown. In the transmit mode, the presence of the body 34 may change capacitance between the transmitter 22 and the receiver 24 and thus change the signal received by the receiver 24. For example, capacitance between the transmitter 22 and the receiver 24 may increase with the presence of the body 34 in the transmit mode. A pattern of sensors 22, 24 may be varied such that the two-dimensional position of the body 34 in the occupant support 10 may be detected. In some embodiments, it may be possible to detect height of the occupant body 34 over the sensors 22, 24 (e.g., determine three-dimensional location of the body 34).

Although illustrated as sensing capacitance with the fringe field 26, it should be understood that in some embodiments capacitance may be measured using changes in the confined field 28 or other near field. For example, a pair of closely associated transmitter 22 and receiver 24 may use the near field to measure mechanical distortion and/or disturbances associated with ballistocardiograph data (e.g., heart rate, breathing rate, and/or blood pressure). The transmitter 22 and the receiver 24 may be positioned close together on the occupant support 10 to enable near field capacitive sensing.

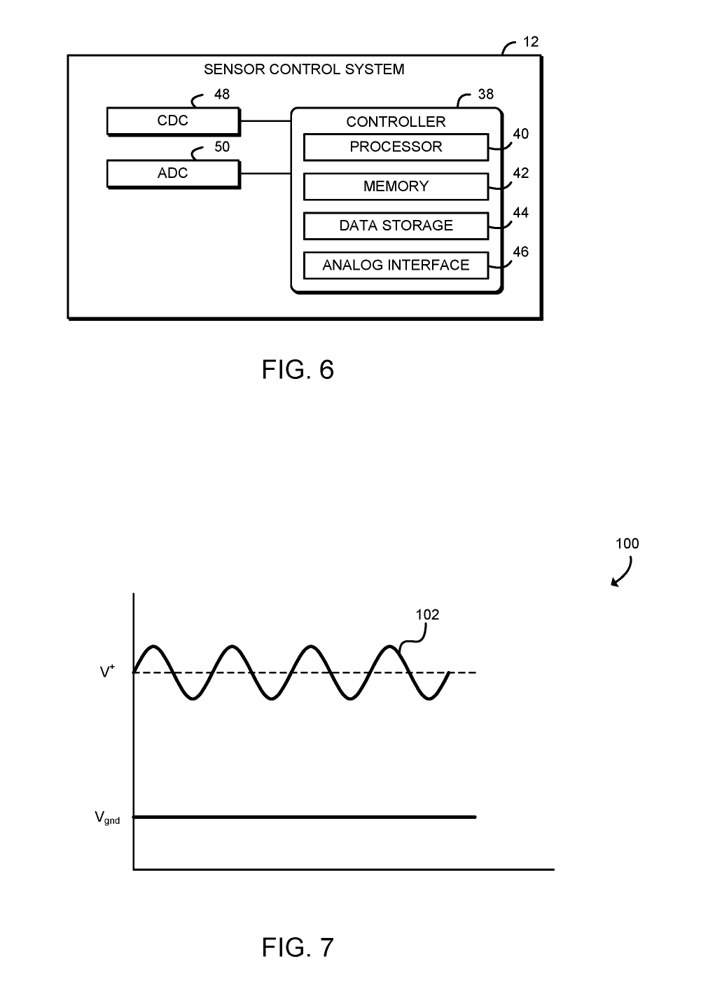

One potential embodiment of the sensor control system 12 is shown in FIG. 6. The sensor control system 12 may be embodied as or otherwise incorporated in any microcontroller, microprocessor, system-on-a-chip (SoC), electronic control unit (ECU), digital signal processor, or other control circuit and related electronics (e.g., analog/digital inputs, signal conditioning stages, amplifiers, and/or other circuitry) capable of performing the operations described herein. Illustratively, the sensor control system 12 includes a controller 38, a capacitance-to-digital converter (CDC) 48, and an analog-to-digital converter (ADC) 50.

The controller 38 may be embodied as a microcontroller, microprocessor, system-on-a-chip (SoC), electronic control unit (ECU), digital signal processor, or other control circuit and related electronics. The controller 38 may be responsible for transmitting the excitation signal to the transmitter 22, receiving a capacitance signal from the receiver 24, and analyzing the capacitance signal to perform occupant detection, biometric measurements, and other operations. To do so, the controller 38 may include a number of electronic components commonly associated with units utilized in the control of electronic and electromechanical systems. For example, the controller 38 may include, amongst other components customarily included in such devices, a processor 40, a memory device 42, and a data storage device 44. The processor 40 may be any type of device capable of executing software or firmware, such as a microcontroller, microprocessor, digital signal processor, or the like. The memory device 42 may be embodied as any type of volatile or non-volatile memory or data storage capable of performing the functions described herein. In operation, the memory device 42 may store various data and software used during operation of the controller 38 such as operating systems, applications, programs, software routines, libraries, and drivers. The data storage device 44 may be embodied as any type of device or devices configured for short-term or long-term storage of data such as, for example, non-transitory, machine-readable media, memory devices and circuits, memory cards, hard disk drives, solid-state drives, non-volatile flash memory, or other data storage devices.

The controller 38 also includes an analog interface circuit 46, which may be embodied as any electrical circuit(s), component, or collection of components capable of performing the functions described herein. The analog interface circuit 46 may convert signals from the processor 40 into output signals which are suitable for presentation to the electrically-controlled components associated with sensor control system 12. For example, the analog interface circuit 46, by use of a variable-frequency signal generator, digital-to-analog (D/A) converter, or the like, may convert digital signals generated by the processor 40 into an excitation signal or other analog signal for transmission by the transmitters 22. Similarly, the analog interface circuit 46 may convert input signals (e.g., from the receivers 24) into signals which are suitable for presentation to an input of the processor 40. In particular, the analog interface circuit 46, by use of a network analyzer, an analog-to-digital converter (ADC), or the like, may convert analog signals into digital signals for use by the processor 40. Additionally, although illustrated as separate components, it is contemplated that, in some embodiments, the analog interface circuit 46 (or portions thereof) may be integrated into the processor 40.

As shown, the sensor control system 12 also includes the CDC 48 and the ADC 50. The CDC 48 may be embodied as an integrated circuit, chip, component, or collection of components capable of measuring capacitance or variations in capacitance of a sensor (e.g., a receiver 24) and outputting that capacitance as a digital value to the controller 38. The CDC 48 may also be capable of generating an excitation signal used to measure capacitance. Similarly, the ADC 50 may be embodied as an integrated circuit, chip, component, or collection of components capable of measuring an analog electrical signal (e.g., voltage or current) and outputting the analog value as a digital value to the controller 38. Although illustrated as separate components, it is contemplated that, in some embodiments, the CDC 48 and/or the ADC 50 (or portions thereof) may be integrated into the controller 38 (e.g., into the analog interface circuit 46 and/or into the processor 40).

Plot 100 illustrates one potential embodiment of an excitation signal 102 as shown in FIG. 7. As shown, the illustrative excitation signal 102 is the combination of an alternating current (AC) waveform superimposed on a positive voltage V⁺. The positive voltage V⁺ is illustrated in comparison to ground voltage V_{gnd}. For example, in some embodiments, V⁺ may be a DC power signal that causes the heating map 20 to generate heat, and the superimposed AC waveform may generate the fringe field 26 used to detect the occupant of the occupant support 10.

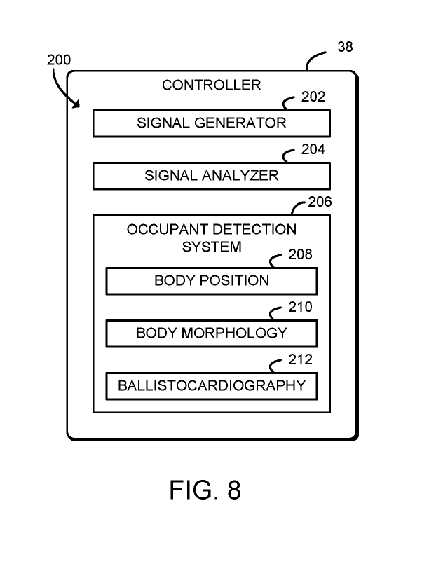

In one illustrative example, the controller 38 establishes an environment 200 during operation as shown in FIG. 8. The illustrative environment 200 includes a signal generator 202, a signal analyzer 204, and an occupant detection system 206. The various components of the environment 200 may be embodied as hardware, firmware, software, or a combination thereof. As such, in some embodiments, one or more of the components of the environment 200 may be embodied as circuitry or collection of electrical devices (e.g., signal generator circuitry 202, signal analyzer circuitry 204, and/or occupant detection circuitry 206). It should be appreciated that, in such embodiments, one or more of the signal generator circuitry 202, the signal analyzer circuitry 204, and/or the occupant detection circuitry 206 may form a portion of one or more of the processor 40, the analog interface circuit 46, and/or other components of the controller 38. Additionally, in some example, one or more of the illustrative components may form a portion of another component and/or one or more of the illustrative components may be independent of one another.

The signal generator 202 is configured to transmit an excitation signal to the transmitters 22. The transmitters 22 and the receivers 24 establish the fringe field 26 in response to transmitting the excitation signal. In some embodiments, the excitation signal may be combined with a power signal to cause a heating mat 20 to generate heat. The signal analyzer 204 is configured to receive a capacitance signal from the receiver 24 in response to transmitting the excitation signal.

The occupant detection system 206 is configured to determine whether an occupant is present in the vehicle seat 10 as a function of the capacitance signal based on capacitive coupling between the occupant and the fringe field 26 in response to receiving the capacitance signal. The presence of the occupant may be determined based on coupling of the fringe field 26 to the body 34 of the occupant in a shunt mode or in a transmit mode, as described further above. The occupant detection system 206 may compare capacitance signals received from multiple receivers 24.

The occupant detection system 206 may be further configured to determine a position of the occupant relative to the vehicle seat 10, which may be a three-dimensional position of the occupant. The occupant detection system 206 may be further configured to determine biometric data associated with the occupant as a function of the capacitance signal. The biometric data may include one or more body morphological measurements of the occupant, or ballistocardiograph data. The ballistocardiograph data may include, for example, heart rate, breathing rate, and blood pressure. A three-dimensional mathematical model may be provided to locate the heart, detect the heart rate, determine heart rate variability, and determine respiration rate. In some embodiments, those functions may be performed by one or more sub-systems, such as a body position subsystem 208, a body morphology subsystem 210, and/or a ballistocardiography subsystem 212.

Occupant detection may differentiate between people and objects, provide for radio frequency identification (RFID), provide for occupant classification for appropriate airbag deployment based on size, weight, and position of an occupant, body mass approximation, and posture assessment. Occupant detection may be incorporated into comfort models regarding contact patterns (e.g., lumbar, upper back adjusters, etc.). Automatic adjustment to fit the occupant's body may also be used in combination with the sensor control system 12.

The following numbered clauses include embodiments that are contemplated and non-limiting:

Clause 1. An occupant support including a vehicle seat that includes a plurality of capacitive sensors.

Clause 2. The occupant support of clause 1, any other clause, or any combination of clauses, wherein the capacitive sensors comprises a first transmitter and a first receiver.

Clause 3. The occupant support of clause 2, any other clause, or any combination of clauses, further comprising a sensor system that comprises a controller.

Clause 4. The occupant support of clause 3, any other clause, or any combination of clauses, wherein the sensor system further comprises an analog interface circuit coupled to the plurality of capacitive sensors.

Clause 5. The occupant support of clause 4, any other clause, or any combination of clauses, wherein the sensor system is configured to transmit an excitation signal to the first transmitter.

Clause 6. The occupant support of clause 5, any other clause, or any combination of clauses, wherein the first transmitter and the first receiver establish a fringe field in response to transmission of the excitation signal.

Clause 7. The occupant support of clause 6, any other clause, or any combination of clauses, the sensor system is further configured to receive a capacitance signal from the first receiver in response to transmission of the excitation signal.

Clause 8. The occupant support of clause 7, any other clause, or any combination of clauses, wherein the sensor system is further configured to determine whether an occupant is present in the vehicle seat as a function of the capacitance signal based on capacitive coupling between the occupant and the fringe field in response to receipt of the capacitance signal.

Clause 9. The occupant support of Clause 8, any other clause, or any combination of clauses, wherein the analog interface circuit comprises an analog to digital converter or a capacitance to digital converter.

Clause 10. The occupant support of Clause 8, any other clause, or any combination of clauses, wherein the vehicle seat comprises a trim layer and a carrier layer, wherein the trim layer covers the carrier layer, and wherein the carrier layer includes the plurality of capacitive sensors.

Clause 11. The occupant support of Clause 10, any other clause, or any combination of clauses, wherein the carrier layer comprises a heat mat that generates heat in response to application of a power signal, wherein the power signal is combined with the excitation signal.

Clause 12. The occupant support of Clause 8, any other clause, or any combination of clauses, wherein each of the plurality of capacitive sensors comprises a piezoelectric film.

Clause 13. The occupant support of Clause 8, any other clause, or any combination of clauses, wherein to determine whether the occupant is present further comprises to determine a position of the occupant relative to the vehicle seat.

Clause 14. The occupant support of Clause 13, any other clause, or any combination of clauses, wherein to determine the position of the occupant comprises to determine a three-dimensional position of the occupant.

Clause 15. The occupant support of Clause 8, any other clause, or any combination of clauses, wherein the sensor system is further configured to determine biometric data associated with the occupant as a function of the capacitance signal.

Clause 16. The occupant support of Clause 15, any other clause, or any combination of clauses, wherein the biometric data comprises a body morphological measurement of the occupant.

Clause 17. The occupant support of Clause 15, any other clause, or any combination of clauses, wherein the biometric data comprises ballistocardiograph data.

Clause 18. The occupant support of Clause 8, any other clause, or any combination of clauses, wherein to determine whether the occupant is present in the vehicle seat as a function of the capacitance signal comprises to determine whether the occupant is present based on coupling of the fringe field to a body of the occupant in a shunt mode or in a transmit mode.

Clause 19. An occupant support comprising a vehicle seat that includes a first transmitter and a first receiver, wherein the first transmitter and the first receiver are positioned close together on the vehicle seat.

Clause 20. The occupant support of Clause 19, any other clause, or any combination of clauses, further comprising a sensor system that comprises a controller and an analog interface circuit coupled to the first transmitter and the first receiver.

Clause 21. The occupant support of Clause 20, any other clause, or any combination of clauses, wherein the sensor system is configured to transmit an excitation signal to the first transmitter.

Clause 22. The occupant support of Clause 21, any other clause, or any combination of clauses, wherein the first transmitter and the first receiver establish a near field in response to transmission of the excitation signal.

Clause 23. The occupant support of Clause 22, any other clause, or any combination of clauses, wherein the sensor system is further configured to receive a capacitance signal from the first receiver in response to transmission of the excitation signal, wherein the capacitance signal fluctuates based on mechanical distortion of the vehicle seat caused by an occupant of the vehicle seat.

Clause 24. The occupant support of Clause 23, any other clause, or any combination of clauses, wherein the sensor system is further configured to determine ballistocardiograph data as a function of the capacitance signal in response to receipt of the capacitance signal.

Clause 25. The occupant support of Clause 24, any other clause, or any combination of clauses, wherein the ballistocardiograph data is indicative of heart rate of the occupant or breathing rate of the occupant.

1. An occupant support comprising:
a vehicle seat that includes a plurality of capacitive sensors, wherein the capacitive sensors comprises a first transmitter and a first receiver; and
a sensor system that comprises a controller and an analog interface circuit coupled to the plurality of capacitive sensors, wherein the sensor system is configured to:
transmit an excitation signal to the first transmitter, wherein the first transmitter and the first receiver establish a fringe field in response to transmission of the excitation signal;
receive a capacitance signal from the first receiver in response to transmission of the excitation signal; and
determine whether an occupant is present in the vehicle seat as a function of the capacitance signal based on capacitive coupling between the occupant and the fringe field in response to receipt of the capacitance signal.

2. The occupant support of claim 1, wherein the vehicle seat comprises a trim layer and a carrier layer, wherein the trim layer covers the carrier layer, and wherein the carrier layer includes the plurality of capacitive sensors.

3. The occupant support of claim 2, wherein the carrier layer comprises a heat mat that generates heat in response to application of a power signal, wherein the power signal is combined with the excitation signal.

4. The occupant support of claim 1, wherein each of the plurality of capacitive sensors comprises a piezoelectric film.

5. The occupant support of claim 1, wherein to determine whether the occupant is present further comprises to determine a position of the occupant relative to the vehicle seat.

6. The occupant support of claim 5, wherein to determine the position of the occupant comprises to determine a three-dimensional position of the occupant.

7. The occupant support of claim 1, wherein the sensor system is further configured to determine biometric data associated with the occupant as a function of the capacitance signal.

8. The occupant support of claim 7, wherein the biometric data comprises a body morphological measurement of the occupant.

9. The occupant support of claim 7, wherein the biometric data comprises ballistocardiograph data.

10. The occupant support of claim 1, wherein to determine whether the occupant is present in the vehicle seat as a function of the capacitance signal comprises to determine whether the occupant is present based on coupling of the fringe field to a body of the occupant in a shunt mode or in a transmit mode.

11. An occupant support comprising:
a vehicle seat that includes a first transmitter and a first receiver, wherein the first transmitter and the first receiver are positioned close together on the vehicle seat; and
a sensor system that comprises a controller and an analog interface circuit coupled to the first transmitter and the first receiver, wherein the sensor system is configured to:
transmit an excitation signal to the first transmitter, wherein the first transmitter and the first receiver establish a near field in response to transmission of the excitation signal;
receive a capacitance signal from the first receiver in response to transmission of the excitation signal, wherein the capacitance signal fluctuates based on mechanical distortion of the vehicle seat caused by an occupant of the vehicle seat; and
determine ballistocardiograph data as a function of the capacitance signal in response to receipt of the capacitance signal.

12. The occupant support of claim 11, wherein the ballistocardiograph data is indicative of heart rate of the occupant or breathing rate of the occupant.
